(19) United States
(12) Patent Application Publication
(10) Pub. No.: US 2014/0303674 A1
(43) Pub. Date: Oct. 9, 2014

(54) PARALLEL ROD CONNECTOR

(71) Applicant: Orthopaedic International, Inc., Cabuyao, Laguna (PH)

(72) Inventor: Jude L. Sasing, Cabuyao (PH)

(21) Appl. No.: 14/358,391

(22) PCT Filed: Nov. 16, 2012
(86) PCT No.: PCT/PH2012/000015

§ 371 Date: May 15, 2014

Related U.S. Application Data
(60) Provisional application No. 61/561,400, filed on Nov. 18, 2011.

(51) Int. Cl. A61B 17/70

(52) U.S. Cl. CPC A61B 17/705
USPC 606/278; 606/279

(57) ABSTRACT

A connector assembly for attaching multiple spinal rods to each other, the connector assembly including a connector body having a top surface, first and second ends, a first opening extending along a longitudinal axis from the first end to the second end, a second opening offset from the first opening and extending along a longitudinal axis from the first end to the second end, and a threaded opening extending along a longitudinal axis from the top surface and into the connector body; and a set screw assembly having a threaded portion that is engageable with the threaded opening of the connector body.

### CROSS REFERENCE TO RELATED APPLICATION

This application claims the benefit under 35 U.S.C. §119(e) of U.S. Provisional Patent Application No. 61/561,400, filed Nov. 18, 2011, and titled “PARALLEL ROD CONNECTOR,” the entirety of which is incorporated herein by reference.

### TECHNICAL FIELD

The present invention relates to spinal implant systems. In particular, the invention relates to connecting two parallel spinal rods to each other via an intermediate connector component.

### BACKGROUND

Many spinal fixation devices have been designed for fusing two or more adjacent vertebrae together. These devices are usually composed of various types of screws and hooks that are attached to each vertebra. The screws and hooks are connected to each other through spinal rods that run roughly parallel to each other along the spine. In certain situations, it is necessary to “extend” the length of a rod in order to accommodate vertebrae in other levels of the spine. This is accomplished by connecting an extension rod parallel to the original rod by using a “domino” connector. The extension rod is placed alongside the main rod, and the domino connector rigidly connects one rod to the other. Domino connectors usually include four fixation set screws to securely fasten the two rods together. Tightening the four set screws can be time consuming and the connector can be bulky in order to accommodate the four set screws. In addition, it can be challenging to tighten each of the four set screws an equal amount to provide uniform engagement between the set screws and the spinal rods. Therefore, there is a need for alternate configurations of spinal fixation systems that provide for relatively easy, secure and accurate attachment of spinal rods to each other.

### SUMMARY

The present invention includes a connector that uses only one set screw, allowing for a faster installation and a smaller overall size. In one aspect of the invention, a connector assembly is provided for attaching multiple spinal rods to each other, which includes a connector body and a set screw assembly. The connector body includes a top surface, first and second ends, a first opening extending along a longitudinal axis from the first end to the second end, a second opening offset from the first opening and extending along a longitudinal axis from the first end to the second end, and a threaded opening extending along a longitudinal axis from the top surface and into the connector body. The set screw assembly includes a threaded portion that is engageable with the threaded opening of the connector. The three longitudinal axes that extend through the connector body can be arranged so that the longitudinal axis of the threaded opening intersects a plane that contains the longitudinal axes of the first and second openings, and the axes of the first and second openings can be parallel to each other. In addition, the longitudinal axis of the threaded opening can be perpendicular to the plane that contains the longitudinal axes of the first and second openings.

In accordance with the invention, the threaded opening of the connector body can include at least one notch that extends from the top surface of the connector body and through a plurality of threads. These notches can engage with a spacer that extends from a distal end of the set screw assembly, via at least one protrusion of the spacer that is engageable with one of the notches of the threaded opening of the connector. The spacer can additionally or alternatively include a wedge-shaped distal surface that is engageable with spinal rods in a rod assembly configuration. The spacer is configured so that the threaded portion of the set screw assembly is rotatable relative to the spacer about the post.

### BRIEF DESCRIPTION OF THE DRAWINGS

The present invention will be further explained with reference to the appended Figures, wherein like structure is referred to by like numerals throughout the several views, and wherein:

FIG. 1 is an exploded perspective view of an embodiment of the invention, including a set screw assembly and a connector;

FIG. 2 is detailed perspective view of the connector of FIG. 1;

FIG. 3 is a cross-sectional side view of the connector of FIGS. 1 and 2;

FIG. 4 is a partially broken away perspective view of the set screw assembly of FIG. 1, including a spacer component;

FIG. 5 is a partially broken away perspective view of a set screw of a set screw assembly of FIG. 4, with the spacer component removed;

FIG. 6 is a perspective view of an embodiment of a spacer of the invention;

FIG. 7 is a perspective view of an embodiment of a spacer of the invention;

FIG. 8 is a perspective view of an embodiment of an assembly of the invention as it can be used for connecting two spinal rods in a generally parallel arrangement;

FIG. 9 is a perspective view of a connector of the invention;

FIG. 10 is an exploded perspective view of another embodiment of the invention, including a set screw assembly and a connector;

FIG. 11 a perspective view of a spacer of the set screw assembly of FIG. 10;

FIG. 12 is an exploded perspective view of another embodiment of the invention, including a set screw assembly and a connector;

FIG. 13 is a perspective view of a spacer of the set screw assembly of FIG. 12;

FIG. 14 is a perspective view of an embodiment of the invention as it is being used for connecting two spinal rods in a generally parallel arrangement; and

FIG. 15 is a perspective view of the assembly of FIG. 14, with the head of the set screw assembly removed.

### DETAILED DESCRIPTION

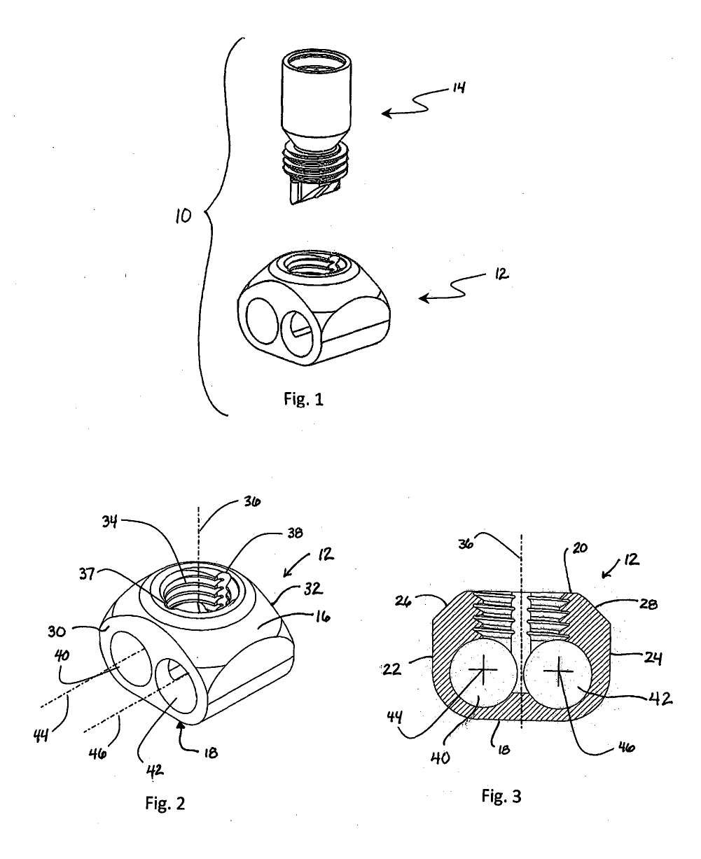

Referring now to the Figures, wherein the components are labeled with like numerals throughout the several Figures, and initially to FIGS. 1-3, a parallel rod assembly 10 of the invention is illustrated, which generally includes a connector 12 and a setscrew assembly 14. The assembly 10 can be used in spinal fixation procedures for attaching two rods together in a predetermined relationship, such as for connecting multiple spinal rods parallel to each other to extend the overall reach of the rods in a spinal application.

Connector 12 includes a body member 16 having a base surface 18 and an opposite top surface 20 that may be parallel or at least slightly angled or otherwise positioned relative to base surface 18. The base surface 18 generally includes a flat or planar portion with curved surfaces extending from its opposite ends and toward top surface 20. Body member 16 further includes opposite sides 22, 24 that extend from the base surface 18 toward top surface 20, and angled surfaces 26, 28 that extend between sides 22, 24 and top surface 20, respectively. In addition, body member 16 includes first and second end surfaces 30, 32 on its opposite ends. The end surfaces 30, 32 may be parallel or at least slightly angled relative to each other. Although many of the various surfaces of body member 16 are illustrated as being generally planar and angled relative to each other, some or all of the surfaces may instead be curved in a convex, concave, or other shaped manner relative to each other to create a desired outer shape for the body member 16. In addition, the overall shape and relative dimensions of the body member 16 can be different from the illustrated embodiment; however, the shape and size of the body member 16 selected should be sufficiently strong to accommodate the various rods and screws that will be inserted therein.

Body member 16 includes a threaded opening 34 that extends from its top surface 20 toward its base surface 18, and first and second openings 40, 42 that extend from first end surface 30 to second end surface 32 (i.e., through the width of the body member 16). The opening 34 further includes one or more notches extending along at least a portion of its length and along the threads, wherein a first notch 37 and a second notch 38 can both be seen in the exemplary configuration of FIG. 2 as being positioned on generally opposite sides of the threaded opening 34. More or less than two of these notches may be provided, depending on the desired engagement with other components in a particular parallel rod assembly. Further, the notches can be arranged in ways other than directly across from each other, and the notches may be evenly or unevenly spaced from each other.

A longitudinal axis 36 extends through threaded opening 34, and longitudinal axes 44, 46 likewise extend through first and second openings 40, 42. In an embodiment of the invention, axis 36 of threaded opening 34 can be arranged to be approximately perpendicular to a plane containing the axes 44, 46 of the first and second openings 40, 42, respectively, and the axes 44, 46 are generally parallel to each other. However, it is possible for the axes of the openings to be at least slightly angled relative to each other and/or for one or both of the axes 44, 46 to be either perpendicular or angled relative to axis 36. In addition, axis 36 is positioned so that it is located at an approximately equal distance from both of the axes 44 and 46, as is shown in FIG. 3, for example.

As can also be seen in FIG. 3, first opening 40 can be generally the same diameter and width as the second opening 42, although it is possible that they have at least slightly different dimensions. In one embodiment, the diameter of each of the openings 40, 42 is large enough to intersect with the threaded opening 34, but small enough that they do not overlap or intersect with each other. In another embodiment, the diameter of the first and second openings can intersect with each other, in which case the diameters of the openings would be larger than the diameters of the rods that will be inserted into them so that the rods do not interfere with each other. In such an embodiment, the rods will have at least a slight amount of additional space to move when the set screw assembly is inserted into the threaded opening to secure the rods. In addition, while illustrated embodiments include circular openings and round rods, the openings and/or rods can have different shapes, such as oval, elliptical, irregular, and the like, wherein such rod shapes may be “wedged” or pushed toward the sides 22, 24 of the connector by an inserted set screw assembly. That is, when the set screw assembly is inserted into a threaded opening of the connector by a certain distance, the outer periphery of the set screw assembly will also extend into both the first and second openings 40, 42, as will be described below relative to an assembly that includes spinal rods.

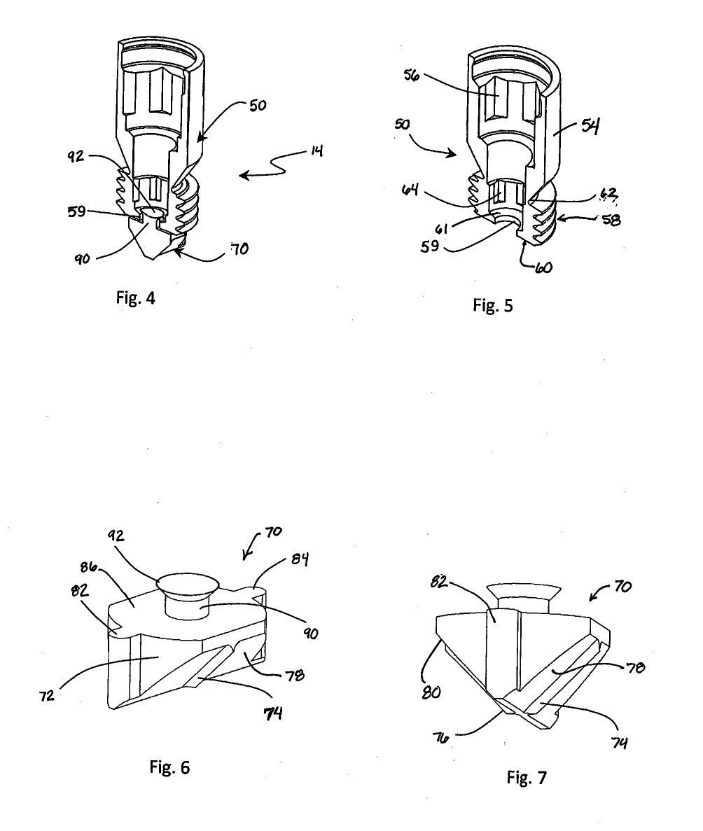

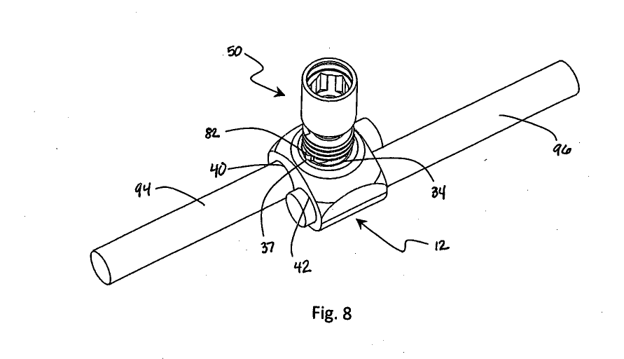

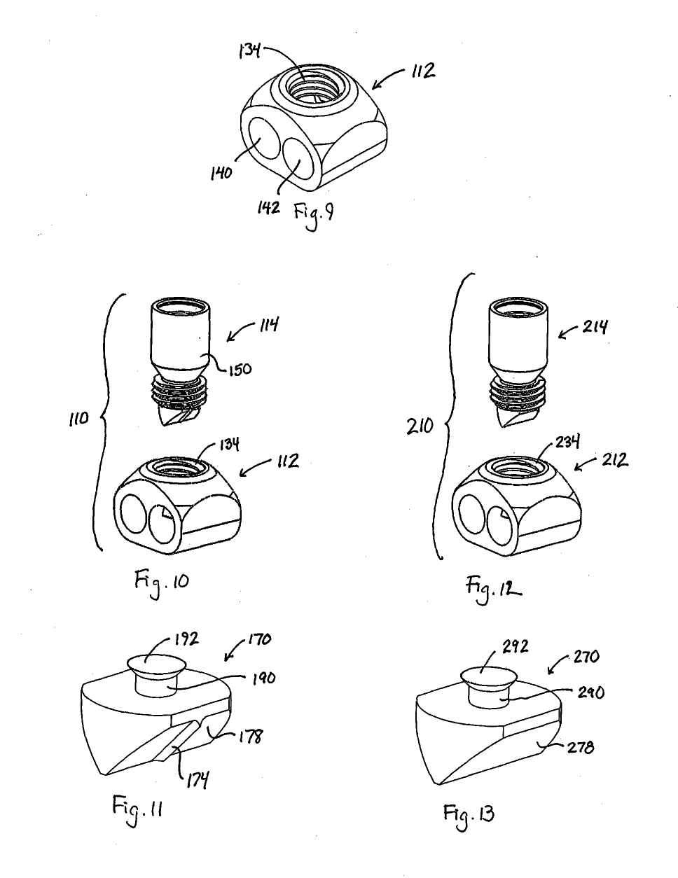

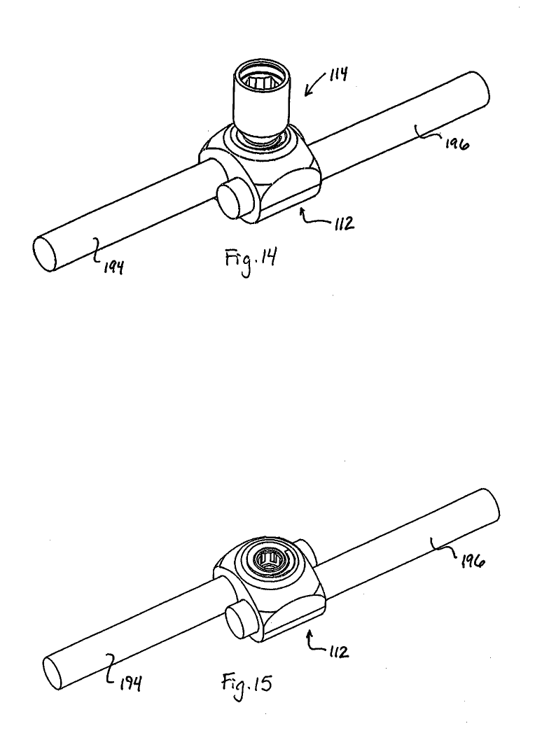

FIG. 4 illustrates a cross-sectional view of set screw assembly 14, which generally includes a set screw 50 and a spacer 70, and set screw 50 is shown without a corresponding spacer 70 in FIG. 5. Although a number of variations of set screw configurations can be used, the illustrated set screw 50 includes a cylindrical head 54 containing a socket 56 that is used for engagement with a tool, such as the hexagonal end of an Allen wrench or other tool, for example. The set screw 50 further includes a threaded portion 58 at its distal end, which may vary in length, but can have a length that is generally selected to correspond with the depth of the threaded opening 34 of the connector 12 with which it will be engaged. The physical properties (e.g., thread angle, pitch, thread depth, and the like) of the external threads of the threaded portion 58 can vary widely, but in any case are selected or designed to engage with the internal threads of the threaded opening 34.

Set screw 50 further includes an aperture 59 at a distal end 60 of the threaded portion 58, which can generally correspond with the center of the distal end 60. The size and shape of aperture 59 is provided for engagement with an extending member of spacer 52, as will be described in further detail below.

A break-off area 62 can optionally be provided between the cylindrical head 54 and the threaded portion 56, which is a section of the set screw 50 that is relatively weak compared to other portions of the set screw 50 so that it will break when subjected to a certain amount of applied torque. In particular, the break-off area 62 can comprise material that is somewhat thinner or otherwise made to be weaker than the adjacent portions of the set screw 50, and/or can be smaller in diameter than the adjacent cylindrical head 54 and threaded portion 58. The break-off area 62 therefore can include features that make the relative strength of this area strong enough that it does not break prematurely, but so that it does not require an undue amount of pressure to break. In operation, the threaded portion 58 of set screw 50 is inserted into the threaded opening of a connector so that their respective threaded portions engage with each other. A tool can then be engaged with the socket 56 of the cylindrical head 54 and rotated until the threads are fully engaged with each other and/or a certain torque is reached. Once this level of engagement is achieved, further rotation of the tool will cause the cylindrical head 54 to break away from the threaded portion 58 at the break-off area 62. Although breaking the cylindrical head 54 from the assembly provides for a lower profile for the final assembly, it is understood that the set screw can optionally omit a break-off area between the two portions such that the cylindrical head 54 remains attached to the threaded portion 58 in the final assembly.

The threaded portion 58 of set screw 50 can further be provided with a socket 64 that has a different size (e.g., smaller) than the socket 56 of cylindrical head 54. The socket 64 is provided for removal of the threaded portion 58 of the set screw 50 from a connector with which it is engaged, such as a relatively small Allen wrench, and this socket 64 will be more readily accessible to the user after the cylindrical head 54 has been removed at the break-off area 62. Alternatively, the set screw 50 may be configured to not have a cylindrical head 54, in which case the set screw 58 is tightened and loosened through socket 64.

FIGS. 6 and 7 illustrate exemplary embodiments of spacer 70, which spacer is also shown as it can be engaged with a set screw 50 in FIG. 4. Spacer 70 includes a body member 72 that includes a roughly wedge-shaped lower surface. Faces of the wedge-shaped portion of spacer 70 optionally include at least one elongated edge protrusion, wherein this embodiment illustrates first and second edge protrusions 74, 76. Each of the edge protrusions 74, 76 can be located approximately in the middle of angled surfaces 78, 80 of the wedge-shaped portion. The spacer 70 further includes at least one protrusion or ear that extends outwardly from a side of the body member 72. In this particular, embodiment, a first protrusion 82 is illustrated as extending from the body member 72 on the generally opposite side of the body member 72 from which a second protrusion 84 extends. The first and second protrusions 82, 84 can be blunt or curved in shape, as shown, or can instead have a different configuration, including different relative sizes, shapes, and the like. In addition, the first and second protrusions 82, 84 can extend the entire distance from a top surface 86 of the body member 72 to the area where angled surfaces 78, 80 meet each other, or the protrusions 82, 84 can instead only extend along a portion of that distance. In any case, the protrusions 82, 84 are designed or selected for engagement within the notches 37, 38 in the threaded opening 34 of the connector 12. That is, the protrusions 82, 84 are shaped, sized, and configured to be able to engage securely with corresponding notches 37, 38.

Spacer 70 further includes a pivot peg 90 extending from the top surface 86 of the body member 72. A flange 92 extends from the end of the peg 90 that is spaced from top surface 86. The flange 92 can be generally circular in shape and tapered toward its top surface, as shown. The size and shape of the peg 90 and flange 92 can vary widely, but can generally be designed or selected to facilitate engagement of the spacer with the threaded portion 58 of set screw 50. With particular reference to FIG. 4, the pivot peg 90 is positioned within the aperture 59 of threaded portion 58, with the flange 92 overlapping onto an inner surface 61 of threaded portion 58 that can best be seen in FIG. 5. Because the diameter of the flange 92 is at least slightly greater than the diameter of aperture 59, the flange 92 will prevent or minimize movement of the spacer 70 vertically to disengage from the threaded portion 58, and will thereby be secured to the threaded portion 58 of set screw 50. That is, spacer 70 is attached to set screw 50 by placing pivot peg 90 inside through aperture 59, and then flaring flange 92 to prevent set screw 50 from separating from spacer 70. It is further noted that because the diameter of the pivot peg 60 is at least slightly smaller than aperture 59, at least a slight lateral movement between spacer 70 and set screw 50 can be possible. The threaded portion 58 of the set screw 50 can be rotated independently from the spacer, such that if the spacer 70 is held in a fixed position, the threaded portion 58 can be rotated about the pivot peg 90.

Referring now to FIG. 8, an exemplary embodiment of an assembly that connects parallel spinal rods using the parallel rod assembly of the invention is illustrated. In operation, set screw assembly 14 is threaded into threaded opening 34 of connector 12 and is oriented so that first protrusion 82 and second protrusion 84 (not visible in this figure) pass into slot 37 and slot 38 (not visible in this figure) of threaded opening 34, respectively. The engagement of the slots and protrusions prevents rotation of the components relative to each other, and provides for a predetermined positioning of these components. First and second spinal rods 94, 96 are then inserted partially into first and second openings 40, 42, respectively, of connector 12. The inner surfaces of the first and second openings 40, 42 can be generally smooth or slightly textured, and are sized and shaped to accept a portion of the length of spinal rods 94, 96, wherein the width of the connector 12 is selected to provide for adequate engagement between the rods 94, 96 and the connector 12 for stability of the rod assembly. Set screw 50 can then be tightened until a desired level of engagement of the threaded portion 58 and the threaded opening 34 is achieved, and then the set screw 50 can be tightened further until the cylindrical head 54 separates from the threaded portion 58 at break-off area 62. In this way, spinal rods 94, 96 will be connected to each other in a generally parallel arrangement via their mutual attachment to connector 12, which attachment is described below in further detail.

Cooperation between the set screw assembly 14 and the connector 12 provides a secure attachment of two rods to a connector with only a single set screw assembly 14. This is accomplished because as set screw 50 is tightened, it pushes the tapered or angled surfaces 78, 80 of spacer 70 downward to engage the spinal rods 94, 96. The lateral movement of spacer 70 relative to set screw 50 allows spacer 70 to adjust so that it is centered between spinal rods 94, 96. With this configuration in which the axis 36 of threaded opening 34 is centered or nearly centered between the openings 40, 42 in which the rods 94, 96 are positioned, when the set screw 50 is tightened, the angled surfaces will remain in contact with the spinal rods 94, 96, and the downward fastening force will be evenly distributed between both spinal rods 94 and 96. When at least one of the angled surfaces 78, 80 includes a protrusion 74, 76, these protrusions can embed into or engage with spinal rods 94 and 96 as spacer 70 is pushed down against the rods. This increases the resistance of spinal rods 94 and 96 to axial motion relative to connector 12, and relative to each other.

Referring now to FIGS. 9-11, another embodiment of a parallel rod assembly 110 of the invention is illustrated, which generally includes a connector 112 and a setscrew assembly 114. The assembly 110 can be used in spinal fixation procedures for attaching two rods together in a predetermined relationship in a manner similar to that described above for parallel rod assembly 10.

Connector 112 is similarly configured to connector 12, in that connector 112 also includes a body member with a threaded opening 134 that extends from its top surface toward its base surface, and first and second openings 140, 142 that extend through the width of the body member. In this embodiment, the opening 134 does not include any notches that extend along at least a portion of its length and along the threads. A longitudinal axis that extends through threaded opening 134 can be arranged to be approximately perpendicular to a plane containing the axes that extend through the first and second openings 140, 142, wherein those axes through openings 140, 142 are generally parallel to each other. However, it is possible for the axes of the openings to be at least slightly angled relative to each other. Furthermore, the axis of opening 134 can be positioned so that it is located at an approximately equal distance from the axes of the first and second openings 140, 142.

Set screw assembly 114, which generally includes a set screw 150 and a spacer 170, is configured for engagement with a tool, such as the hexagonal end of an Allen wrench or other tool, for example. The set screw 150 further includes a threaded portion at its distal end, which may vary in length, but can have a length that is generally selected to correspond with the depth of the threaded opening 134 of the connector 112 with which it will be engaged. The physical properties (e.g., thread angle, pitch, thread depth, and the like) of the external threads of the threaded portion can vary widely, but in any case are selected or designed to engage with the internal threads of the threaded opening 134.

Spacer 170, which is shown in detail in FIG. 11, includes a body member that is roughly wedge-shaped. Spacer 170 optionally includes at least one edge protrusion, such as protrusion 174, which extends from an angled surface 178. The edge protrusions can be located approximately in the middle of angled surfaces their respective angled surfaces. The spacer 170 does not include any protrusions or ears that extend outwardly from the body member of the type that are described relative to spacer 70 to provide the additional orienting and movement control of the spacer 70 described above; however, the operation of the assembly will be essentially the same as described above relative to other embodiments. Spacer 170 further includes a pivot peg 190 extending from a top surface of the body member, and a flange 192 extending from the end of the peg 190. The flange 192 can be a circular disk and can be tapered toward its top surface, as shown. The size and shape of the peg 190 and flange 192 can vary widely, but can generally be designed or selected to facilitate engagement of the spacer with the threaded portion of set screw 150.

Referring now to FIGS. 14 and 15, and initially to FIG. 14, an embodiment of an assembly that connects parallel spinal rods using the connector 112 and set screw assembly 114 is illustrated. In operation, set screw assembly 114 is threaded into threaded opening 134 of connector 112 and is oriented so that the wedge shaped portion of spacer 170 is positioned with each of its angled surfaces facing one of two spinal rods 194, 196, which are inserted into first and second openings of connector 112. The upper area of the set screw assembly can then be tightened until a desired level of engagement of its threaded portion and the threaded opening 134 is achieved, and then the set screw 150 can optionally be tightened further until its cylindrical head breaks away from its threaded portion, as is illustrated in FIG. 15. In this way, spinal rods 194, 196 will be connected to each other in a generally parallel arrangement via their mutual attachment to connector 112.

Again, cooperation between the set screw assembly 114 and the connector 112 provides a secure attachment of two rods to a connector with only a single set screw assembly 114. This is accomplished because as set screw 150 is tightened, it pushes the tapered or angled surfaces of spacer 170 downward to engage the spinal rods 194, 196. The lateral movement of spacer 170 relative to set screw 150 allows spacer 170 to adjust so that it is centered between spinal rods 194, 196. With this configuration, the downward fastening force will be evenly distributed between spinal rods 194 and 196. When at least one of the angled surfaces 78, 80 includes a protrusion (e.g., protrusion 174), these protrusions can embed into or engage with spinal rods 194 and 196 as spacer 170 is pushed down against the rods. This increases the resistance of spinal rods 194 and 196 to axial motion relative to connector 112, and relative to each other.

FIGS. 12 and 13 illustrate an assembly 210 that is similar to that discussed herein relative to FIGS. 10 and 11; however, assembly 210 includes a spacer 270 that does not include any protrusions from its angled surfaces, such as angled surface 178. However, the spacer 270 may still include a pivot peg 290 and upper flange member 292 to provide secure engagement between the spacer 270 of a set screw assembly 214. The set screw assembly 214 can be assembled to a connector 212 in a similar manner to that described above relative to spacers that also include a protrusion.

While the embodiments illustrated show the rods 94, 96, 194, 196 as having approximately the same diameters, the present invention can also be applied to rods of different diameters. In this case, holes 40, 42, 140, 142 can have different dimensions to match the dimensions of the corresponding rods.

The present invention has now been described with reference to several embodiments thereof. The entire disclosure of any patent or patent application identified herein is hereby incorporated by reference. The foregoing detailed description and examples have been given for clarity of understanding only. No unnecessary limitations are to be understood therefrom. It will be apparent to those skilled in the art that many changes can be made in the embodiments described without departing from the scope of the invention. Thus, the scope of the present invention should not be limited to the structures described herein, but only by the structures described by the language of the claims and the equivalents of those structures.

1. A connector assembly for attaching multiple spinal rods to each other, the connector assembly comprising:
a connector body comprising a top surface, first and second ends, a first opening extending along a longitudinal axis from the first end to the second end, a second opening offset from the first opening and extending along a longitudinal axis from the first end to the second end, and a threaded opening extending along a longitudinal axis from the top surface and into the connector body; and
a set screw assembly comprising a threaded portion that is engageable with the threaded opening of the connector body.

2. The connector assembly of claim 1, wherein the longitudinal axis of the threaded opening intersects a plane that contains the longitudinal axes of the first and second openings.

3. The connector assembly of claim 2, wherein the axes of the first and second openings are parallel to each other.

4. The connector assembly of claim 3, wherein the longitudinal axis of the threaded opening is perpendicular to the plane that contains the longitudinal axes of the first and second openings.

5. The connector assembly of claim 2, wherein the longitudinal axis of the threaded opening is located between and at an approximately equal distance from the longitudinal axes of the first and second openings.

6. The connector assembly of claim 1, wherein the threaded opening of the connector body comprises at least one notch that extends from the top surface of the connector body and through a plurality of internal threads positioned within the threaded opening.

7. The connector assembly of claim 6, wherein the set screw assembly further comprises a spacer extending from its distal end, and wherein the spacer comprises at least one protrusion that is engageable with one of the notches of the threaded opening of the connector body.

8. The connector assembly of claim 6, wherein the threaded opening of the connector body comprises two notches that are positioned on opposite sides of the threaded opening.

9. The connector assembly of claim 1, wherein the set screw assembly further comprises a spacer extending from its distal end, the spacer comprising a wedge-shaped distal surface.

10. The connector assembly of claim 9, wherein the spacer further comprises a post extending from a top surface of the spacer, and a flange extending from an end of the post, and wherein the threaded portion of the set screw assembly is rotatable relative to the spacer about the post.

11. The connector assembly of claim 1, wherein the set screw assembly further comprises a cylindrical head extending proximally from the threaded portion, wherein the cylindrical head comprises an internal tool engagement aperture.

12. The connector assembly of claim 11, wherein threaded portion of the set screw assembly comprises external threads and an internal tool engagement aperture that has a different configuration than a configuration of the internal tool engagement aperture of the cylindrical head.

13. The connector assembly of claim 1, wherein the set screw assembly further comprises a cylindrical head spaced from the threaded portion by a break-off area.

14. The connector assembly of claim 1, in combination with first and second spinal rods, wherein the first spinal rod is insertable into the first opening and the second spinal rod is insertable into the second opening.

15. The combination of claim 14, wherein the set screw assembly comprises a spacer extending from its threaded portion, the spacer comprising first and second surfaces arranged in a wedge-shaped configuration, and wherein the first and second surfaces contact a portion of the first and second rods, respectively, when the threaded portion of the set screw assembly is engaged with the threaded opening of the connector body.

16. A connector for attaching multiple spinal rods to each other, the connector comprising a top surface, first and second ends, a first opening extending along a longitudinal axis from the first end to the second end, a second opening offset from the first opening and extending along a longitudinal axis from the first end to the second end, and a threaded opening extending along a longitudinal axis from the top surface and into the connector, wherein the threaded opening is configured for engagement with a threaded portion of a set screw assembly.

17. A method of connecting two spinal rods to each other, comprising the steps of:
inserting a first spinal rod into a first opening of a connector body and inserting a second spinal rod into a second opening of a connector body, wherein the first opening extends along a longitudinal axis of the connector body from its first end to its second end, and wherein the second opening is offset from the first opening and extends along a longitudinal axis from the first end to the second end,
inserting a threaded portion of a set screw assembly into a threaded opening extending along a longitudinal axis of the connector body from its top surface and into the connector body, wherein the set screw assembly comprises a spacer at its distal end; and
rotating the threaded portion of the set screw assembly into the threaded opening of the connector body to secure the first and second spinal rods to the connector body.

18. The method of claim 17, wherein the step of rotating the threaded portion comprises maintaining the spacer in a fixed rotational position relative to the spacer body while the threaded portion is being rotated.

19. The method of claim 17, wherein the step of inserting the threaded portion of the set screw assembly into the threaded opening of the connector body further comprises engaging at least one protrusion of the spacer with at least one notch that extends through multiple threads of the threaded opening.

20. The method of claim 17, wherein the step of rotating the threaded portion of the set screw assembly further comprises rotating the threaded portion until a predetermined torque is achieved, and then further rotating the threaded portion until a portion of the set screw assembly separates from the threaded portion.
